(19) United States
(12) Patent Application Publication
(10) Pub. No.: US 2020/0397058 A1
(43) Pub. Date: Dec. 24, 2020

(54) MODULAR VAPORIZER

(71) Applicants: Daniel James Combs, Centerville, OH (US); Cody Badder, Greenville, MI (US)

(72) Inventors: Daniel James Combs, Centerville, OH (US); Cody Badder, Greenville, MI (US)

(21) Appl. No.: 16/445,561

(22) Filed: Jun. 19, 2019

(51) Int. Cl. A24F 47/00; H05B 3/40

(52) U.S. Cl. CPC A24F 47/008; A61M 11/042; H05B 3/40

(57) ABSTRACT

A modular vaporizer includes a battery, a heating element, a materials compartment, and a mouthpiece. The modular vaporizer further includes a plurality of modules stacked one on top of the other. At least a first module of the plurality of modules includes at least one electrical rod, and at least a second module of the plurality of modules includes at least one electrical rod slot. The at least one electrical rod is releasably disposed within the at least one electrical rod slot, establishing an electrical connection between the first module and the second module.

### BACKGROUND OF THE INVENTION

The present invention relates to a modular vaporizer and, more particularly, to a modularized vaporizer pen that allows a user to interchange different types of components.

A vaporizer, colloquially known as a vape, is a device used to vaporize substances for inhalation. Plant substances can be used, commonly cannabis, tobacco, or other herbs or blends. However, they can also be filled with a combination propylene glycol, glycerin, and drugs such as nicotine or tetrahydrocannabinol as a liquid solution.

Vaporizers contain various forms of extraction chambers including straight bore, venturi, or sequential venturi, and are made of materials such as metal or glass. The extracted vapor may be collected in an inflatable bag or inhaled directly through a hose or pipe. When used properly, cooler temperatures due to lack of combustion result in significantly more efficient extraction of the ingredients. Hence, the irritating and harmful effects of smoking are heavily reduced, as is its secondhand smoke.

Vaporizer pens or e-cigarettes are a handheld battery-powered vaporizer that simulates smoking by providing some of the behavioral aspects of smoking, including the hand-to-mouth action of smoking, but without combusting tobacco or marijuana. Typically, vaporizer pens include a mouthpiece and a battery. The mouthpiece includes a heating element and an oil chamber. The mouthpiece connects to the battery usually via mechanical threads.

As can be seen, there is a need for a modularized vaporizer pen that allows a user to interchange different types of components.

### SUMMARY OF THE INVENTION

In one aspect of the present invention, a modular vaporizer comprises: a battery; a heating element; a materials compartment; a mouthpiece; and a plurality of modules stacked one on top of the other, wherein at least a first module of the plurality of modules comprises at least one electrical rod, and at least a second module of the plurality of modules comprises at least one electrical rod slot wherein the at least one electrical rod is releasably disposed within the at least one electrical rod slot, establishing an electrical connection between the first module and the second module.

These and other features, aspects and advantages of the present invention will become better understood with reference to the following drawings, description and claims.

### BRIEF DESCRIPTION OF THE DRAWINGS

FIG. 1 is a perspective view of an embodiment of the present invention;

FIG. 2 is a front view of an embodiment of the present invention;

FIG. 3 is a top view of an embodiment of the present invention;

FIG. 4 is a top perspective exploded view of an embodiment of the present invention;

FIG. 5 is a bottom perspective exploded view of an embodiment of the present invention;

FIG. 6 is a section view of the present invention taken along line 6-6 in FIG. 3;

FIG. 7 is a section view of the present invention taken along line 7-7 in FIG. 3;

FIG. 8 is a section view of the present invention taken along line 8-8 in FIG. 3;

FIG. 9 is a detail section view of an embodiment of the present invention; and

FIG. 10 is a schematic view of an embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

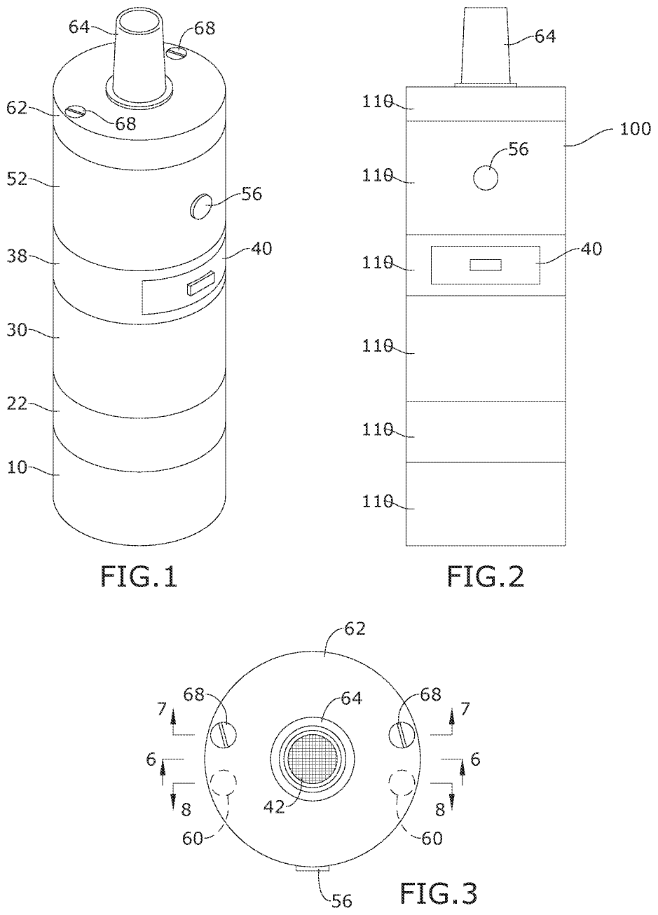

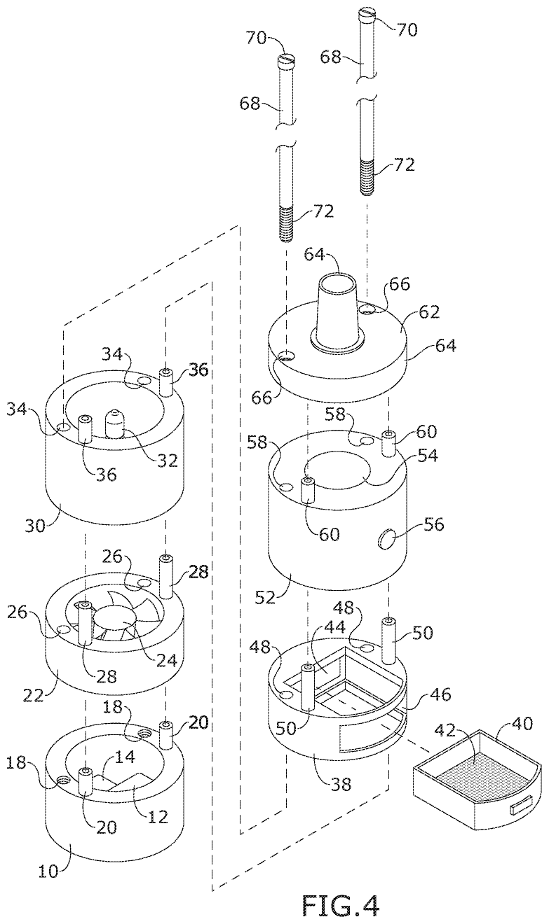

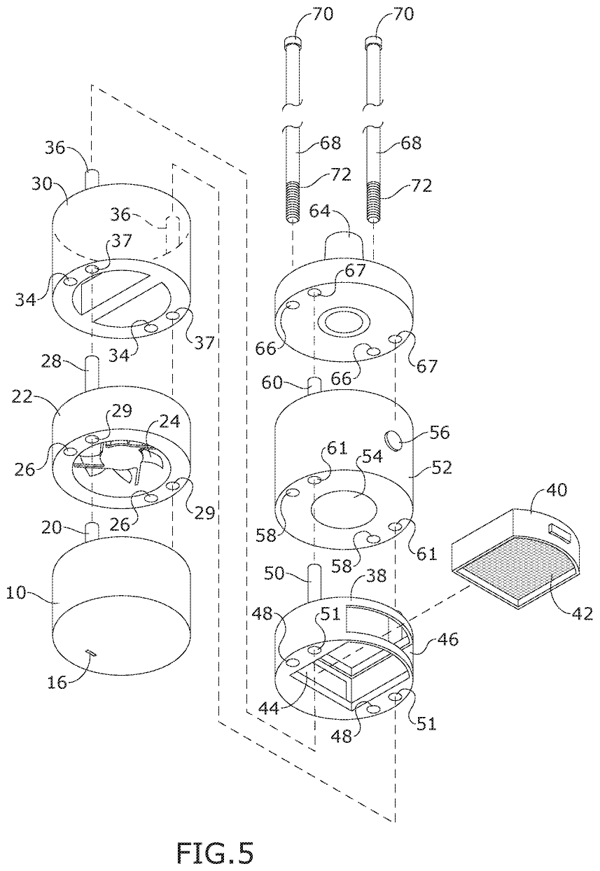

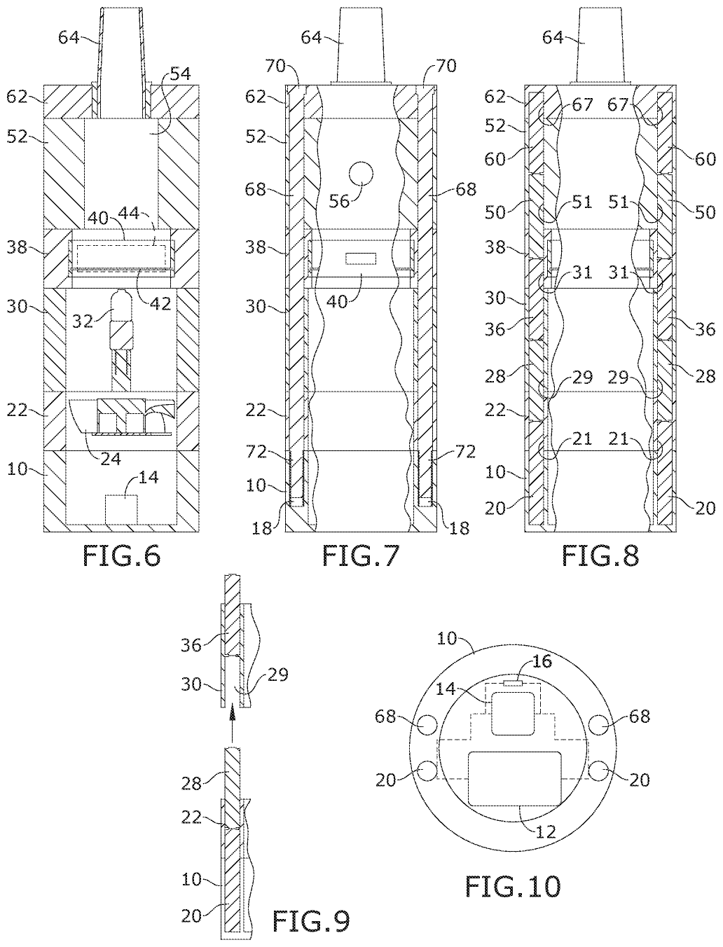

Referring to FIGS. 1 through 10, the present invention includes a modular vaporizer 100. The modular vaporizer 100 includes a battery 12, a heating element 32, a materials compartment 40, and a mouthpiece 64. The modular vaporizer 100 further includes a plurality of modules 110 stacked one on top of the other. At least a first module of the plurality of modules 110 includes at least one electrical rod, and at least a second module of the plurality of modules 110 includes at least one electrical rod slot. The at least one electrical rod is releasably disposed within the at least one electrical rod slot, establishing an electrical connection between the first module and the second module.

Each of the plurality of modules 110 may include a hollow cylinder. Each module 110 can vary in length from about 0.5 inches to about 82 inches. In certain embodiments, the modules 110 may include two electrical rods extending from a top and two electrical rod slots defined on the bottom. The slots and rods may be disposed across from each other within the rim of the module 110. The electrical rods transmit electricity power from one module 110 to the next. The modules may be made out of glass, titanium, steel, or the like. Some modules 110 may include electrical components protected or layered to ensure safety and operational use. Thin metal, steel, and titanium of perforated sheets may be utilized to provide the compartment design and also act as filters for vapor air paths.

In certain embodiments, the first module is a battery module 10 including the battery 12. The battery 12 may be electrically connected to a power input port 16 which may be electrically connected to a wall socket by a power cord for recharging the battery 12. The battery 12 provides the main power source to the present invention and the battery module 10 acts as a base. The battery 12 transmits the electricity via electrical rods 20. A voltage regulator 14 charges the conducting rods 20. In certain embodiments, the battery module 10 may include a microprocessor to perform specific tasks such as controlling the heating element 32 after receiving data from a temperature sensor. In such embodiments, the electrical rods may be used for power transfer and communication. The battery module 10 may include a pair of electrical rods 20 extending from the top.

In certain embodiments, one of the plurality of modules is a fan module 22 with a fan 24. The fan module 24 may include a pair of electrical rod slots 29 defined on the bottom and a pair of electrical rods 28 protruding from the top. The pair of electrical rods 20 of the battery module 10 fit within the pair of electrical rod slots 29 of the fan module 22, electrically connecting the battery 12 to the fan 24 and thereby powering the fan 24.

In certain embodiments, the second module is a heating element module 30 including the heating element 32. The heating element module 30 may further include a pair of electrical rod slots 37 defined on the bottom and a pair of electrical rods 36 protruding from the top. The pair of electrical rods 28 of the fan module 22 fit within the pair of electrical rod slots 37 of the heating element module 30, electrically connecting the battery 12 to the heating element 32 and thereby powering the heating element 32. In certain embodiments, the heating element 32 is a Halogen bulb(S). The heating element 32 heats the entity being vaporized. The light dimmer switch controls the heating element. Halogen wires may be hooked into the dimmer via jumper wires.

In certain embodiments, the materials compartment 40 may be part of a materials compartment module 38. The materials compartment module 38 includes a pair of electrical rod slots 51 defined on the bottom and a pair of electrical rods 50 protruding from the top. The pair of electrical rods 36 of the heating element module 30 fit within the pair of electrical rod slots 51 of the materials compartment module 38 electrically connecting the battery 12 to the pair of electrical rods 36 for continuing the power stream. The materials compartment 40 includes a removeable drawer. The removeable drawer includes a screen 42. The materials compartment 40 may further include a magnet 44 releasably retaining the removeable drawer within a drawer slot 46. A user may place a plant or oil material within the drawer. The heating element 32 is disposed below the drawer and heats/vaporizes the contents within the drawer.

In certain embodiments, one of the plurality of modules 110 is an air chamber module 52. The air chamber module 52 includes an air chamber 54 and a power button 56. The air chamber module 52 includes a pair of electrical rod slots 61 defined on the bottom and a pair of electrical rods 60 protruding from the top. The pair of electrical rods 50 of the materials compartment module 38 fit within the pair of electrical rod slots 61 of the air chamber module 52 electrically connecting the battery 12 to the power button 56.

Another of the plurality of modules 110 is a mouthpiece module 62 including the mouthpiece 64. The mouthpiece module 62 includes a pair of electrical rod slots 67 defined on the bottom. The pair of electrical rods 60 of the air chamber module 52 fit within the pair of electrical rod slots 67 of the mouthpiece module 64, connecting the mouthpiece module 62 to the air chamber module 52. The mouthpiece 64 may be a perforated metal, titanium, or steel sheet.

To use the present invention, a user may press the power button 56, which turns on the heating element 32 and fan 24 to vaporize the contents within the drawer. The user may inhale through the mouthpiece 64 and the vapor may travel through the air chamber 54, through the mouthpiece 64 and into the user's lungs.

The plurality of modules may be further secured together by a pair of elongated rods 68 having heads 70 and threaded ends 72. The battery module 10 may include threaded openings 18, the fan module 22 may include clearance openings 26, the heating element module 30 may include clearance openings 34, the materials compartment module 38 may include clearance openings 48, the air chamber module 52 may include clearance openings 58 and the mouthpiece module 52 may include countersunk openings 66. The pair of elongated rods 68 fit through the clearance openings 26, 34, 48, 58 and screw into the threaded openings 18 of the battery module 10. The heads 70 are driven into the countersunk openings 66, preventing the modules 110 from detaching from one another.

It should be understood, of course, that the foregoing relates to exemplary embodiments of the invention and that modifications may be made without departing from the spirit and scope of the invention as set forth in the following claims.

What is claimed is:

1. A modular vaporizer comprising:
a battery;
a heating element;
a materials compartment;
a mouthpiece; and
a plurality of modules stacked one on top of the other, wherein
at least a first module of the plurality of modules comprises at least one electrical rod, and
at least a second module of the plurality of modules comprises at least one electrical rod slot wherein
the at least one electrical rod is releasably disposed within the at least one electrical rod slot, establishing an electrical connection between the first module and the second module.

2. The modular vaporizer of claim 1, wherein the at least one electrical rod comprises a pair of electrical rods protruding from a top of the first module, and the at least one electrical rod slot is a pair of rod slots defined on a bottom of the second module.

3. The modular vaporizer of claim 1, wherein the first module is a battery module comprising the battery and the second module is a heating element module comprising the heating element.

4. The modular vaporizer of claim 3, wherein one of the plurality of modules is a materials compartment module comprising the materials compartment, and another of the plurality of modules is a mouthpiece module comprising the mouthpiece, wherein the heating element module further comprises at least one electrical rod, the materials compartment module comprises at least one electrical rod slot and at least one electrical rod, and the mouthpiece module comprises at least one electrical rod slot.

5. The modular vaporizer of claim 3, wherein one of the plurality of modules is an air chamber module comprising an air chamber and a power button, wherein the air chamber module comprises at least one electrical rod slot and at least one electrical rod.

6. The modular vaporizer of claim 3, wherein one of the plurality of modules is a fan module comprising a fan, wherein the fan module comprises at least one electrical rod slot and at least one electrical rod.

7. The modular vaporizer of claim 1, wherein the materials compartment comprises a removeable drawer, the removeable drawer comprising a screen.

8. The modular vaporizer of claim 7, wherein the materials compartment further comprises a magnet releasably retaining the removeable drawer within a drawer slot.

9. The modular vaporizer of claim 1, wherein the plurality of modules are connected together by an elongated rod running through aligning openings of the plurality of modules.

10. The modular vaporizer of claim 9, wherein the elongated rod comprises a head and a threaded end, wherein one of the plurality of modules comprises a countersunk opening, and another of the plurality of modules comprises a threaded opening.
